(19) United States
(12) Patent Application Publication
(10) Pub. No.: US 2004/0048860 A1
(43) Pub. Date: Mar. 11, 2004

(54) Use of selective noradrenaline reuptake inhibitors for the treatment of tension-type headache

(76) Inventors: Jes Olesen, Hellerup (DK); Rigmor Jensen, Virum (DK); Lars Bendtsen, Slagelse (DK)

Correspondence Address: BIRCH STEWART KOLASCH & BIRCH, PO BOX 747, FALLS CHURCH, VA, 22040-0747, US

(21) Appl. No.: 10/415,588

(22) PCT Filed: Oct. 29, 2001
(86) PCT No.: PCT/DK01/00717

(30) Foreign Application Priority Data
Oct. 31, 2000 (DK) PA 2000 01638

(51) Int. Cl. A61K 31/537

(52) U.S. Cl. 514/238.8

(57) ABSTRACT

This invention relates to the use of selective noradrenaline reuptake inhibitors, in particular reboxetine, for the treatment of tension-type headache.

### TECHNICAL FIELD

This invention relates to the use of selective noradrenaline reuptake inhibitors, such as reboxetine, for the treatment of tension-type headache.

### BACKGROUND ART

Previously, headache disorders were not clearly distinguished and it was widely believed that they formed part of a continuum and were strongly related. In 1988, The International Headache Society, (IHS) via its ad hoc committee on classification published a document on Classification and Diagnostic Criteria for Headache Disorders, Cranial Neuralgias and Facial Pain. A new entity was here defined by name of tension-type headache.

Tension-type headache was subdivided by the IHS Classification Committee into an episodic form occurring less than half of all days and a chronic form occurring half of all days or more. Furthermore, both of these divisions were further subdivided into a form with disorder of pericranial muscle and a form without such disorder.

The classification and diagnostic criteria for tension-type headache are explained in further details in WO 98/19674 (which is hereby incorporated by reference).

Epidemiological studies done by the inventors have shown that chronic tension-type headache affects three percent of the population at any given time, the lifetime prevalence being as high as six percent. Severe episodic tension-type headache defined as persons having headache twice a week or more occurs in approximately ten percent of the population. Thus, tension-type headache is a serious problem with significant socioeconomic implications, involving enormous loss of workdays and quality of life.

Infrequent episodic tension-type headaches are usually cured by aspirin or paracetamol. However, the more frequent and severe types of episodic tension-type headache often do not respond well to plain analgesics and the patients are left virtually without effective pharmacotherapy. In chronic tension-type headache, sufferers face therapeutic problems are of two kinds: Firstly, the great majority of these patients have no effect of plain analgesics and get no other therapy. Secondly, because of desperation these individuals often overconsume plain analgesics. Chronic headache is the most common reason for excessive use of plain analgesics.

Amitriptyline is the only drug with a proven prophylactic effect in chronic tension-type headache, but it helps only a minority of the patients and it only reduces headache by 30%. Furthermore, it has many side effects, such as sedation and dryness of mouth.

WO 98/19674 describes a method for treating tension-type headache by interacting with neuronal transmission in relation to pain in connection with headache in a way that prevents or decreases central-sensitization.

However, there is a continued strong interest in the development of a more selective and effective therapy with fewer side effects for the treatment of patients with tension-type headache.

### SUMMARY OF THE INVENTION

According to the invention it has now been found that selective noradrenaline reuptake inhibitors (selective NRI's) can be used for the treatment of tension-type headache.

Thus, in its first aspect, the invention relates to the use of a selective NRI or a pharmaceutically acceptable salt or a prodrug thereof for the manufacture of a medicament for the treatment, prevention or alleviation of tension-type headache in a subject.

In another aspect the invention relates to a method of treatment, prevention or alleviation of tension-type headache in a subject, which method comprises administering to said subject a therapeutically effective amount of a selective NRI or a pharmaceutically acceptable salt or a prodrug thereof.

Other objects of the invention will be apparent to the person skilled in the art from the following detailed description and examples.

### DETAILED DISCLOSURE OF THE INVENTION

The present invention provides the use of a selective noradrenaline reuptake inhibitor (selective NRI) or a pharmaceutically acceptable salt or a prodrug thereof for the manufacture of a medicament for the treatment, prevention or alleviation of tension-type headache in a subject.

In a further aspect the invention provides a method of treatment, prevention or alleviation of tension-type headache in a subject, which method comprises administering to said subject a therapeutically effective amount of a selective NRI or a pharmaceutically acceptable salt or a prodrug thereof.

The subject to be treated according to this invention is a living body, preferably a mammal, most preferably a human, in need for such treatment.

In one embodiment, the selective NRI is a compound represented by formula I

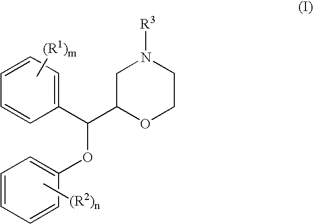

wherein

m and m are independently 1, 2, or 3;

each of R¹ and R² independently of each are selected from the group consisting of

hydrogen, halogen, hydroxy, C₁₋₆-alkoxy,

C₁₋₆-alkyl, optionally substituted with one or more hydroxy, halogen, or C₁₋₆-alkoxy,

phenyl-C₁₋₆-alkyl, phenyl-C₁₋₆-alkoxy, wherein the phenyl is optionally substituted with one or more halogen, C₁₋₆-alkyl, C₁₋₆-alkoxy, or hydroxy; and wherein the C₁₋₆-alkyl is optionally substituted with one or more halogen;

R³ is selected from the group consisting of

hydrogen,

C₁₋₆-alkyl, optionally substituted with one or more halogen, hydroxy, or C₁₋₆-alkoxy,

C₂₋₄-alkenyl, C₂₋₄-alkynyl,

phenyl-C₁₋₄-alkyl, wherein the phenyl is optionally substituted with one or more C₁₋₆-alkyl, halogen, hydroxy, or C₁₋₆-alkoxy; and wherein the C₁₋₆-alkyl is optionally substituted with one or more halogen,

C₃₋₇-cycloalkyl, optionally substituted with one or more C₁₋₆-alkyl, halogen, hydroxy or C₁₋₆-alkoxy; and wherein the C₁₋₆-alkyl is optionally substituted with one or more halogen;

or an enantiomer or a mixture of its enantiomers thereof or a pharmaceutically acceptable salt or a prodrug thereof.

In a further embodiment, the selective NRI is a compound represented by the formula I wherein

m and n are independently 1, or 2;

each of R¹ and R² independently of each other are selected from the group consisting of

hydrogen, methoxy, ethoxy, chlorine, and trifluoromethyl;

R³ is selected from the group consisting of

hydrogen, methyl, and isopropyl.

In a special embodiment, m is 1. In a further special embodiment, R¹ is hydrogen. In a still further embodiment, n is 1. In a further embodiment, R² is C₁₋₆-alkoxy, such as ethoxy. In a still further embodiment, R³ is hydrogen.

The above compounds of formula I and a process for their preparation are described in U.S. Pat. No. 4,229,449.

In a still further embodiment the selective NRI is

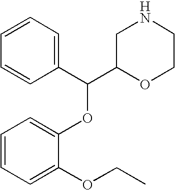

or an enantiomer or a mixture of its enantiomers thereof or a pharmaceutically acceptable salt or a prodrug thereof.

In a further embodiment, the selective RNI is reboxetine or a pharmaceutically acceptable salt thereof.

In a still further embodiment of the invention the tension-type headache to be treated, prevented or alleviated is of the type chronic tension-type headache.

Selective Noradrenaline Reuptake Inhibitors (Selective NRI's)

In the context of this invention, a selective NRI is a compound that selectively acts at the noradrenaline reuptake site, thereby resulting in an increased level of noradrenaline in the peripheral and/or central nervous system. Essentially the selective NRI does not affect the-metabolism of e.g. serotonin.

The potential of a given compound to act as a selective NRI may be determined, using standard in vitro binding assays and/or standard in vivo functionality tests.

One specific example of a selective NRI is reboxetine, 2-[α-(2-ethoxy)-phenoxy-benzyl]morpholine. Reboxetine is usually administered as the racemate. The racemate or either enantiomer can be used in the form of a pharmaceutical salt, such as an acid addition salt, or the free base of the molecule. A specific example of addition salts of reboxetine is reboxetine methansulfonate.

Definition of Substituents

In the context of this invention halogen represents a fluorine, a chlorine, a bromine or an iodine atom.

In the context of this invention a C₁₋₆-alkyl group designates a univalent saturated, straight or branched hydrocarbon chain containing from one to six carbon atoms, including pentyl, isopentyl, neopentyl, tertiary pentyl, hexyl and isohexyl. In a preferred embodiment a C₁₋₆-alkyl group represents a C₁₋₄-alkyl group, including butyl, isobutyl, secondary butyl, and tertiary butyl. In another preferred embodiment of this invention a C₁₋₆-alkyl group represents a C₁₋₃-alkyl group, which may in particular be methyl, ethyl, propyl or isopropyl.

In the context of this invention an C₂₋₄-alkenyl group designates a carbon chain comprising of from two to four carbon atoms and containing one or more double bonds. In a preferred embodiment the C₂₋₄-alkenyl group of the invention is ethenyl; 1- or 2-propenyl; 1-, 2- or 3-butenyl, or 1,3-butenyl.

In the context of this invention an C₂₋₄-alkynyl group designates a carbon chain of from two to four carbon atoms and containing one or more triple bonds. In a preferred embodiment the C₂₋₄-alkynyl group is ethynyl; 1-, or 2-propynyl; or 1-, 2-, or 3-butynyl, or 1,3-butynyl.

In the context of this invention a C₃₋₇-cycloalkyl group designates a cyclic alkyl group containing of from three to seven carbon atoms, including cyclopropyl, cyclobutyl, cyclopentyl, cyclohexyl and cycloheptyl.

In the context of this invention a C₁₋₆-alkoxy group designates an “C₁₋₆-alkyl-O—” group, wherein C₁₋₆-alkyl is as defined above.

Pharmaceutically Acceptable Salts

The chemical compound of the invention may be provided in any form suitable for the intended administration. Suitable forms include pharmaceutically (i.e. physiologically) acceptable salts, and pre- or prodrug forms of the chemical compound of the invention.

Examples of pharmaceutically acceptable addition salts include, without limitation, the non-toxic inorganic and organic acid addition salts such as the hydrochloride derived from hydrochloric acid, the hydrobromide derived from hydrobromic acid, the nitrate derived from nitric acid, the perchlorate derived from perchloric acid, the phosphate derived from phosphoric acid, the sulphate derived from sulphuric acid, the formate derived from formic acid, the acetate derived from acetic acid, the aconate derived from aconitic acid, the ascorbate derived from ascorbic acid, the benzenesulphonate derived from benzensulphonic acid, the benzoate derived from benzoic acid, the cinnamate derived from cinnamic acid, the citrate derived from citric acid, the embonate derived from embonic acid, the enantate derived from enanthic acid, the fumarate derived from fumaric acid, the glutamate derived from glutamic acid, the glycolate derived from glycolic acid, the lactate derived-from lactic acid, the maleate derived from maleic acid, the malonate derived from malonic acid, the mandelate derived from mandelic acid, the methanesulphonate derived from methane sulphonic acid, the naphthalene-2-sulphonate derived from naphtalene-2-sulphonic acid, the phthalate derived from phthalic acid, the salicylate derived from salicylic acid, the sorbate derived from sorbic acid, the stearate derived from stearic acid, the succinate derived from succinic acid, the tartrate derived from tartaric acid, the toluene-p-sulphonate derived from p-toluene sulphonic acid, and the like. Such salts may be formed by procedures well known and described in the art.

Metal salts of a chemical compound of the invention includes alkali metal salts, such as the sodium salt of a chemical compound of the invention containing a carboxy group.

Other acids such as oxalic acid, which may not be considered pharmaceutically acceptable, may be useful in the preparation of salts useful as intermediates in obtaining a chemical compound of the invention and its pharmaceutically acceptable acid addition salt.

In the context of this invention the “onium salts” of N-containing compounds are also contemplated as pharmaceutically acceptable salts. Preferred “onium salts” include the alkyl-onium salts, the cycloalkyl-onium salts, and the cycloalkylalkyl-onium salts.

The chemical compound of the invention may be provided in dissoluble or indissoluble forms together with a pharmaceutically acceptable solvents such as water, ethanol, and the like. Dissoluble forms may also include hydrated forms such as the monohydrate, the dihydrate, the hemihydrate, the trihydrate, the tetrahydrate, and the like. In general, the dissoluble forms are considered equivalent to indissoluble forms for the purposes of this invention.

Prodrugs

The substance used according to, the invention may be administered as such or in the form of a suitable prodrug thereof. The term “prodrug” denotes a bioreversible derivatve of the drug, the bioreversible derivative being therapeutically substantially inactive per se but being able to convert in the body to the active substance by an enzymatic or non-enzymatic process.

Thus, examples of suitable prodrugs of the substances used according to the invention include compounds obtained by suitable bioreversible derivatization of one or more reactive or derivatizable groups of the parent substance to result in a bioreversible derivative. The derivatization may be performed to obtain a higher bioavailability of the active substance, to stabilize an otherwise unstabl active substance, to increase the lipophilicity of the substance administered, etc.

Examples of types of substances which may advantageously be administered in the form of prodrugs are carboxylic acids, other acidic groups and amines, which may be rendered more lipophilic by suitable bioreversible derivatization. As examples of suitable groups may be mentioned bioreversible esters or bioreversible amides. Amino acids are typical examples of substances which, in their unmodified form, may have a low absorption upon administration. Suitable prodrug derivatives of amino acids will be one or both of the above-mentioned types of bioreversible derivatives.

Steric Isomers

The chemical compounds of, the present invention may exist in (+) and (−) forms as well as in racemic forms. The racemates of these isomers and the individual isomers themselves are within the scope of the present invention.

Racemic forms can be resolved into the optical antipodes by known methods and techniques. One way of separating the diastereomeric salts is by use of an optically active acid, and liberating the optically active amine compound by treatment with a base. Another method for resolving racemates into the optical antipodes is based upon chromatography on an optical active matrix. Racemic compounds of the present invention can thus be resolved into their optical antipodes, e.g., by fractional crystallisation of d- or I-(tartrates, mandelates, or camphorsulphonate) salts for example.

The chemical compounds of the present invention may also be resolved by the formation of diastereomeric amides by reaction of the chemical compounds of the present invention with an optically active activated-carboxylic acid such as that derived from (+) or (−) phenylalanine, (+) or (−) phenylglycine, (+) or (−) camphanic acid or by the formation of diastereomeric carbamates by reaction of the chemical compound of the present invention with an optically active chloroformate or the like.

Additional methods for the resolving the optical isomers are known in the art. Such methods include those described by Jaques J. Collet A, & Wilen S in “Enantiomers, Racemates, and Resolutions”, John Wiley and Sons, New York (1981).

Optical active compounds can also be prepared from optical active starting materials.

Pharmaceutical Compositions

In another aspect the invention provides novel pharmaceutical compositions comprising a therapeutically effective amount of the chemical compound, of the invention.

While a chemical compound of the invention for use in therapy may be administered in the form: of the raw chemical compound, it is preferred to introduce the active ingredient, optionally in the form of, a physiologically acceptable salt, in a pharmaceutical composition together with one or more adjuvants, excipients, carriers, buffers, diluents, and/or other customary pharmaceutical auxiliaries.

In a preferred embodiment, the invention provides pharmaceutical compositions comprising the chemical compound of the invention, or a pharmaceutically acceptable salt or derivative thereof, together with one or more pharmaceutically acceptable carriers therefor, and, optionally, other therapeutic and/or prophylactic ingredients. The carrier(s) must be “acceptable” in the sense of being compatible with the other ingredients of the formulation and not harmful to the recipient thereof.

Pharmaceutical compositions of the invention may be those suitable for oral, rectal, bronchial, nasal, topical (including buccal and sub-lingual), transdermal, vaginal or parenteral (including cutaneous, subcutaneous, intramuscular, intraperitoneal, intravenous, intraarterial, intracerebral, intraocular injection or infusion) administration, or those fin a form suitable for administration by inhalation or insufflation, including powders and liquid aerosol administration, or by sustained release systems. Suitable examples, of sustained release systems include semipermeable matrices of solid hydrophobic polymers containing the compound of the invention, which matrices may be in form of shaped articles, e.g. films or microcapsules.

The chemical compound of the invention, together with a conventional adjuvant, carrier, or diluent, may thus be placed into the form of pharmaceutical comrpositions and unit dosages thereof. Such forms include solids, and in particular tablets, filled capsules, powder and pellet forms, and liquids, in particular aqueous or non-aqueous solutions, suspensions, emulsions, elixirs, and capsules filled with the same, all for oral use, suppositories for rectal administration, and sterile injectable solutions for parenteral use. Such pharmaceutical compositions and unit dosage forms thereof may comprise conventional ingredients in conventional proportions, with or without additional active compounds or principles, and such unit dosage forms may contain any suitable effective amount of the active ingredient commensurate with the intended daily dosage range to be employed.

The chemical compound of the present invention can be administered in a wide variety of oral and parenteral dosage forms. It will be obvious to those skilled in the art that the following dosage forms may comprise, as the active component, either a chemical compound of the invention or a pharmaceutically acceptable salt of a chemical compound of the invention.

For preparing pharmaceutical compositions from a chemical compound of the present invention, pharmaceutically acceptable carriers can be either solid or liquid. Solid form preparations include powders, tablets, pills, capsules, cachets, suppositories, and dispersible granules. A solid carrier can be one or more substances which may also act as diluents, flavouring agents, solubilizers, lubricants, suspending agents, binders, preservatives, tablet disintegrating agents, or an encapsulating material.

In powders, the carrier is a finely divided solid, which is in a mixture with the finely divided active component.

In tablets, the active component is mixed with the carrier having the necessary binding capacity in suitable proportions and compacted in the shape and size desired.

The powders and tablets preferably contain from five or ten to about seventy percent of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as carrier providing a capsule in which the active component, with or without carriers, is surrounded by a carrier, which is thus in association with it. Similarly, cachets and lozenges are included. Tablets, powders, capsules, pills, cachets, and lozenges can be used as solid forms suitable for oral administration.

For preparing suppositories, a low melting wax, such as a mixture of fatty acid glyceride or cocoa butter, is first melted and the active component is dispersed homogeneously therein, as by stirring. The molten homogenous mixture is then poured into convenient sized moulds, allowed to cool, and thereby to solidify.

Compositions suitable for vaginal administration may be presented as pessaries, tampons, creams, gels, pastes, foams or sprays containing in addition to the active ingredient such carriers as are known in the art to be appropriate liquid preparations include solutions, suspensions, and emulsions, for example, water or water-propylene glycol solutions. For example, parenteral injection liquid preparations can be formulated as solutions in aqueous polyethylene glycol solution.

The chemical compound according to the present invention may thus be formulated for parenteral administration (e.g. by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, and may contain formulation agents such as suspending, stabilising and/or dispersing agents. Alternatively, the active ingredient may be in powder form, obtained by aseptic isolation of sterile solid or by lyophilization from solution, for constitution with a suitable vehicle, e.g. sterile, pyrogen-free water, before use.

Aqueous solutions suitable for oral use can be prepared by dissolving the active component in water and adding suitable colorants, flavours, stabilising and thickening agents, as desired.

Aqueous suspensions suitable for oral use can be made by dispersing the finely divided active component in water with viscous material, such as natural or synthetic gums, resins, methylcellulose, sodium carboxymethylcellulose, or other well known suspending agents.

Also included are solid form preparations, intended for conversion shortly before use to liquid form preparations for oral administration. Such liquid forms include solutions, suspensions, and emulsions. In addition to the active component such preparations may comprise colorants, flavours, stabilisers, buffers, artificial and natural sweeteners, dispersants, thickeners, solubilizing agents, and thee like.

For topical administration to the epidermis the chemical compound of the invention may be formulated as ointments, creams or lotions, or as a transdermal patch. Ointments and creams may, for example, be formulated with an aqueous or oily base with the addition of suitable thickening and/or gelling agents. Lotions may be formulated with an aqueous or oily base and will in general also contain one or more emulsifying agents, stabilising agents, dispersing agents, suspending agents, thickening agents, or colouring agents.

Compositions suitable for topical administration in the mouth include lozenges comprising the active agent in a flavoured base, usually sucrose and acacia or tragacanth; pastilles comprising the active ingredient in an inert base such as gelatin and glycerine or sucrose and acacia; and mouthwashes comprising the active ingredient in a suitable liquid carrier.

Solutions or suspensions are applied directly to the nasal cavity by conventional means, for example with a dropper, pipette or spray. The compositions may be provided in single or multi-dose form.

Administration to the respiratory tract may also be achieved by means of an aerosol formulation in which the active ingredient is provided in a pressurised pack with a suitable propellant such as a chlorofluorocarbon (CFC) for example dichlorodifluoromethane, trichlorofluoromethane, or dichlorotetrafluoroethane, carbon dioxide, or other suitable gas. The aerosol may conveniently also contain a surfactant such as lecithin. Th dos of drug may be controlled by provision of a metered valve.

Alternatively the active ingredients may be provided in the form of a dry powder, for example a powder mix of the compound in a suitable powder base such as lactose, starch, starch derivatives such as hydroxypropylmethyl cellulose and polyvinylpyrrolidone (PVP). Conveniently the powder carrier will form a gel in the nasal cavity. The powder composition may be presented in unit dose form for example in capsules or cartridges of, e.g., gelatin, or blister packs from which the powder may be administered by means of an inhaler.

In compositions intended for administration to the respiratory tract, including intranasal compositions, the compound will generally have a small particle size for example of the order of 5 microns or less. Such a particle size may be obtained by means known in the art, for example by micronization.

When desired, compositions adapted to give sustained release of the active ingredient may be employed.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packaged tablets, capsules, and powders in vials or ampoules. Also, the unit dosage form can be a capsule, tablet, cachet, or lozenge itself, or it can be the appropriate number of any of these in packaged form.

Tablets or capsules for oral administration and liquids for intravenous administration and continuous infusion are preferred compositions.

Further details on techniques for formulation and administration may be found in the latest edition of Remington's Pharmaceutical Sciences (Maack Publishing Co., Easton, Pa.).

A therapeutically effective dose refers to that amount of active ingredient, which ameliorates the symptoms or condition. Therapeutic efficacy and toxicity, e.g. ED₅₀ and LD₅₀, may be determined by standard pharmacological procedures in cell cultures or experimental animals. The dose ratio between'therapeutic and toxic effects is the therapeutic index and may be expressed by the ratio LD₅₀/ED₅₀. Pharmaceutical compositions exhibiting large therapeutic indexes are preferred.

The dose administered must of course be carefully adjusted to the age, weight and condition of the individual being treated, as well as the route of administration, dosage form and regimen, and the result desired, and the exact dosage should of course be determined by the practitioner.

The actual dosage depend on the nature and severity of the disease being treated and the route of administration, and is within the discretion of the physician, and may be varied by titration of the dosage to the particular circumstances of this invention to produce the desired therapeutic effect. However, it is presently contemplated that pharmaceutical compositions containing of from about 0.01 to about 500 mg of active ingredient per individual dose, preferably of from about 0.1 to about 100 mg, most preferred of from about 1 to about 10 mg, are suitable for therapeutic treatments.

The active ingredient may be administered in one or several doses per day. A satisfactory result can, in certain instances, be obtained at a dosage as low as 0.01 μg/kg i.v and 0.1 μg/kg p.o. The upper limit of the dosage range is presently considered to be about 10 mg/kg i.v. and 100 mg/kg p.o. Preferred ranges are from about 0.1 μg/kg to about 10 mg/kg/day i.v., and from about 1 μg/kg to about 100 mg/kg/day p.o.

Methods of Therapy

The efficacy of use of the selective NRI according to the invention can be evaluated by standard in vivo studies as e.g. described by Bendtsen L, Jensen R, Olesen J, in J Neurol Neurosurg Psychiatry, 1996, 61:285-290.

### EXAMPLES

The invention is further illustrated with reference to the following examples, which are not intended to be in any way limiting to the scope of the invention as claimed.

### Example 1

Prophylactic Treatment with Reboxetine

Prophylactic treatment with reboxetine has been given to 10 patients with chronic tension-type headache treated at the outpatient headache clinic at the Department of Neurology, Glostrup Hospital, University of Copenhagen, Denmark. There were 5 women and 5 men with a mean age of 44 years. All patients had been suffering from chronic tension-type headache for several years, and had been treatment resistant to other headache prophylactics. Before treatment, the patents underwent a general and a neurological examination and completed a diagnostic headache diary during a four-week run-in period.

The patients filled in a headache diary before and during treatment recording headache characteristics, accompanying symptoms and intake of analgesics.

Results

In total, 10 patents have been treated with reboxetne (as the methanesulphonate salt). The initial dose was 4 mg once daily and the final dose was 8 mg once daily, which was reached in 5 out of the 6 patients that completed the treatment series. The patents were instructed to take the tablet two to three hours before bedtime. Four patients stopped treatment within the first 4 weeks due to side effects. Headache frequency was reduced with 10%, whereas there was a pronounced reduction of the other treatment variables (AUC (intensity×duration) with 47%, in intensity with 78% and in consumption of analgesics with 55%). Efficacy was estimated to be very good in 66% of the patients that fulfilled the treatment series. Sleeping problems and dizziness were the most common side effect, and four patients dropped out because of these side effects. No serious side effects were reported.

1. The use of a selective noradrenaline reuptake inhibitor (selective NRI) or a pharmaceutically acceptable salt or a prodrug thereof for the manufacture of a medicament for the treatment, prevention or alleviation of tension-type headache in a subject.

2. The use according to claim 1 wherein the selective NRI is a compound represented by formula I 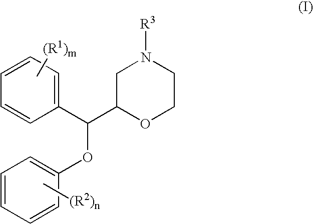
wherein
m and m are independently 1, 2, or 3;
each of R¹ and R² independently of each are selected from the group consisting of
hydrogen, halogen, hydroxy, C₁₋₆-alkoxy,
C₁₋₆-alkyl, optionally substituted with one or more hydroxy, halogen, or C₁₋₆-alkoxy,
phenyl-C₁₋₆-alkyl, phenyl-C₁₋₆-alkoxy, wherein the phenyl is optionally substituted with one or more halogen, C₁₋₆-alkyl, C₁₋₆-alkoxy, or hydroxy; and wherein the C₁₋₆-alkyl is optionally substituted with one or more halogen;
R³ is selected from the group consisting of
hydrogen,
C₁₋₆-alkyl, optionally substituted with one or more halogen, hydroxy, or C₁₋₆-alkoxy,
C₂₋₄-alkenyl, C₂₋₄-alkynyl,
phenyl-C₁₋₆-alkyl, wherein the phenyl is optionally substituted with one or more C₁₋₆-alkyl, halogen, hydroxy, or C₁₋₆-alkoxy; and wherein the C₁₋₆-alkyl is optionally substituted with one or more halogen,
C₃₋₇-cycloalkyl, optionally substituted with one or more C₁₋₆-alkyl, halogen, hydroxy or C₁₋₆-alkoxy; and wherein the C₁₋₆-alkyl is optionally substituted with one or more halogen;
or an enantiomer or a mixture of its enantiomers thereof or a pharmaceutically acceptable salt-or a prodrug thereof for the manufacture of a medicament for the treatment, prevention or alleviation of tension-type headache in a subject.

3. The use according to claim 2, wherein the selective NRI is a compound represented by the formula I wherein
m and n are independently 1, or 2;
each of R¹ and R² independently of each other are selected from the group consisting of hydrogen, methoxy, ethoxy, chlorine, and trifluoromethyl;
R³ is selected from the group consisting of hydrogen, methyl, and isopropyl.

4. The use according to claim 3, wherein the selective RNI is 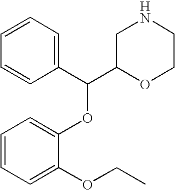
or an enantiomer or a mixture of its enantiomers thereof or a pharmaceutically acceptable salt or a prodrug thereof.

5. The use according to claim 4, wherein the selective RNI is reboxetine or a pharmaceutically acceptable salt thereof

6. The use according to any one of the claims 1-5 wherein the tension-type headache to be treated, prevented, or alleviated is of the type chronic tension-type headache.

7. A method of treatment, prevention or alleviation of tension-type headache in a subject, which method comprises administering to said subject a therapeutically effective amount of a selective NRI or a pharmaceutically acceptable salt or a prodrug thereof.
